(19) United States
(12) Patent Application Publication
(10) Pub. No.: US 2011/0137235 A1
(43) Pub. Date: Jun. 9, 2011

(54) ULTRASONIC CANCER THERAPY ACCELERATOR

(75) Inventors: Koki Kanehira, Fukuoka (JP); Shuji Sonezaki, Fukuoka (JP); Yumi Ogami, Fukuoka (JP); Toshiaki Banzai, Tokyo (JP); Tomomi Nakamura, Fukuoka (JP)

(73) Assignee: TOTO Ltd., Kitakyushu-chi, Fukuoka (JP)

(21) Appl. No.: 13/057,858

(22) PCT Filed: Aug. 7, 2009
(86) PCT No.: PCT/JP2009/064042

§ 371 Date: Feb. 23, 2011

(30) Foreign Application Priority Data
Aug. 8, 2008 (JP) 2008-205233

(51) Int. Cl. A61M 37/00; C07F 7/28; C07F 19/00; C12N 13/00; A61K 31/28; A61P 35/00

(52) U.S. Cl. 604/22; 556/54; 556/55; 556/31; 435/173.1; 514/492; 428/402

(57) ABSTRACT

Provided is an ultrasonic cancer therapy accelerator comprising titanium oxide-metal complex particles showing a long-lasting antitumor effect imparted thereto while sustaining the dispersibility and catalytic activity thereof which are obtained by dispersing titanium oxide-metal complex particles in an aqueous solvent with the use of a water-soluble polymer and modifying the same with molecules containing a low-valent transition metal via linker molecules having been bound thereto without denaturing the water-soluble polymer. To the titanium oxide surface of titanium oxide-metal complex particles which have been dispersed in an aqueous solvent with the use of a water-soluble polymer, linker molecules are bound via at least one functional group selected from the group consisting of carboxyl, amino, diol, salicylate and phosphate groups followed by the modification with low-valent transition metal-containing molecules via the linker molecules. Thus, an ultrasonic cancer therapy accelerator, which comprises titanium oxide-metal complex particles showing a long-lasting antitumor effect imparted thereto while sustaining the dispersibility and catalytic activity thereof, can be provided. This ultrasonic cancer therapy accelerator, which accumulates in an affected area, can be used as a drug for therapy combined with ultrasonic irradiation.

### TECHNICAL FIELD

The present invention relates to an ultrasonic cancer therapy accelerator which comprises titanium oxide-metal complex particles characterized in that titanium oxide complex particles are dispersed with the use of a water soluble polymer in an aqueous solvent and to the surface of the the titanium oxide linker molecules are bound, without denaturing a water soluble polymer, and further a low-valent transition metal is bound to the titanium oxide complex particles through the linker molecules, wherein the titanium oxide-metal complex particles have catalytic activity upon exposure to ultrasonic waves and also have a persistent antitumor effect.

### BACKGROUND ART

Titanium oxide is said to have an isoelectric point at a pH value around 6. Thus, titanium oxide particles are disadvantageously coagulated in a near-neutral aqueous solvent, making it very difficult to evenly disperse the titanium oxide particles in the aqueous solvent. Accordingly, various attempts have been made to evenly disperse titanium oxide particles in an aqueous dispersant.

It is known that PEG (polyethylene glycol) is added as a dispersant to improve the dispersibility of the titanium oxide particles in a dispersion medium (see patent document 1 (JP H2(1990)-307524 A) and patent document 2 (JP 2002-60651 A)).

Surface-modified titanium oxide fine particles obtained by strongly binding a hydrophilic polymer such as polyacrylic acid or polyethylene glycol to a titanium oxide surface in titanium oxide fine particles through a functional group such as a carboxyl or diol group are also known (see patent document 3 (WO 2004/087577) and patent document 4 (JP 2008-162995 A)). According to these techniques, since there is no possibility that the surface of titanium oxide is all covered as a result of polymerization between functional groups, the particles have good dispersibility even in near-neutral physiological saline close to in-vivo environments and also exert catalytic activity upon exposure to ultraviolet light or ultrasonic waves.

Further, ultrasonic cancer therapy accelerators that exert catalytic activity upon exposure to ultrasonic waves have been proposed (see patent document 5 (JP 2008-094824 A)). According to this technique, upon exposure of titanium oxide-containing metal semiconductor particles to ultrasonic waves, an antitumor effect attained by the generation of radical species or active oxygen species can be expected while ensuring high level of safety. In this case, it is considered that the life time of radical species such as hydroxy radicals is so short that, immediately after the stop of ultrasonic irradiation, the amount of radical species generated until then is significantly reduced.

Various methods are considered effective in generating hydroxy radicals. For example, a Fenton reaction in which hydrogen peroxide is decomposed through a Haber-Weiss mechanism using a low-valent transition metal such as divalent iron is well known (see non-patent document 1).

On the other hand, a material of titanium oxide complexed with iron has been proposed. For example, an attempt to recover complexed titanium oxide fine particles by magnetic field from a solution through the utilization of magnetic properties of iron has been proposed. Specifically, examples of proposals include a system comprising titanium oxide supported on the surface of a ferromagnetic metal (see patent document 6 (JP H9(1997)-66237 A)), a system comprising titanium oxide supported on the surface of a soft magnetic powder (see patent document 7 (JP 2000-288404 A)), and a system comprising a photocatalyst supported on the surface of ferrite magnetic particles (see patent document 8 (JP H11-156200 A)). An additional proposal is to complex titanium oxide or tungsten oxide with iron from the viewpoint of enhancing the efficiency of charge separation (see patent document 9 (JP 2006-198465 A)).

These materials of titanium oxide complexed with iron, however, have been studied with a view to enhancing or utilizing a function of titanium oxide as a photocatalyst, and no reference is made to catalytic activity exerted upon exposure to ultrasonic waves. Further, no study has been made on stable dispersibility in in-vivo environments. Furthermore, no study has been made on the utilization of a Fenton reaction.

### PRIOR ART DOCUMENTS

Patent Documents

Patent document 1: JP H2(1990)-307524 A

Patent document 2: JP 2002-60651 A

Patent document 3: WO 2004/087577

Patent document 4: JP 2008-162995 A

Patent document 5: JP 2008-094824 A

Patent document 6: JP H9(1997)-66237 A

Patent document 7: JP 2000-288404 A

Patent document 8: JP H11-156200 A

Patent document 9: JP 2006-198465 A

Non-Patent Document

Non-patent document 1: Kassei Sanso Shu no Kagaku (Chemistry of Active Oxygen Speceies) [Quaternaly Chemical Review No. 7] edited by The Chemical Society of Japan

### SUMMARY OF INVENTION

The present inventors have now found that, when linker molecules are bound to a titanium oxide surface of titanium oxide complex particles dispersed in an aqueous solvent with the use of a water soluble polymer through at least one functional group selected from the group consisting of carboxyl, amino, diol, salicylate and phosphate groups, molecules containing a low-valent transition metal can be additionally bound, while maintaining dispersibility and catalytic activity without denaturing the water soluble polymer.

Accordingly, an object of the present invention is to provide an ultrasonic cancer therapy accelerator comprising titanium oxide-metal complex particles showing a long-lasting antitumor effect imparted thereto while sustaining the dispersibility and catalytic activity thereof which are obtained by dispersing titanium oxide-metal complex particles, which have antitumor effect utilizing catalytic activity exerted by ultrasonic irradiation, in an aqueous solvent with the use of a water-soluble polymer and modifying the titanium oxide-metal complex particles in the dispersed state with molecules containing a low-valent transition metal via linker molecules having been bound thereto without denaturing the water-soluble polymer.

Thus, according to the present invention, an ultrasonic cancer therapy accelerator that are titanium oxide-metal complex particles which can maintain a high level of dispersibility without denaturing a water soluble polymer, have catalytic activity upon exposure to ultrasonic waves, and have persistent antitumor effect imparted thereto can be provided by binding linker molecules to a titanium oxide surface in titanium oxide complex particles dispersed in an aqueous solvent with the use of the water soluble polymer and further binding low-valent transition metal-containing molecules through the linker molecules. Irradiation of the ultrasonic cancer therapy accelerator with ultrasonic waves can realize persistant generation of radicals even after the stop of the ultrasonic irradiation by a Fenton reaction between hydrogen peroxide accumulated in the system and molecules containing a low-valent transition metal bound to the ultrasonic cancer therapy accelerator, and the continuous generation of the radicals can provide a persistent antitumor effect. A high antitumor effect can be attained by administering the ultrasonic cancer therapy accelerator to an organism to allow the ultrasonic cancer therapy accelerator to be accumulated at a portion around cancer, which is an affected part, by EPR effect relying upon the size of particles and applying ultrasonic waves. Therefore, the ultrasonic cancer therapy accelerator of the present invention can be utilized as an agent that can accelerate ultrasonic cancer therapy conducted by accumulating an accelerator at an affected part and further applying ultrasonic waves.

Thus, according to the present invention, there is provided an ultrasonic cancer therapy accelerator comprising titanium oxide-metal complex particles that have catalytic activity upon exposure to ultrasonic waves, the titanium oxide-metal complex particles comprising:

titanium oxide complex particles comprising titanium oxide particles and a water soluble polymer bound to the surface of the titanium oxide particles through at least one functional group selected from the group consisting of carboxyl, amino, diol, salicylate and phosphate groups; and linker molecules further bound to the surface of the titanium oxide complex particles, the linker molecules being a compound
(1) having at least one functional group selected from the group consisting of carboxyl, amino, diol, salicylate and phosphate groups and
(2) comprising a) a saturated or unsaturated chain hydrocarbon group having 6 to 40 carbon atoms, b) a substituted or unsubstituted saturated or unsaturated five- or six-membered heterocyclic group, or c) a substituted or unsubstituted saturated or unsaturated five- or six-membered cyclohydrocarbon group,
the functional groups in the compound not being mutually polymerized, the compound being bound to the titanium oxide through the functional group,

a low-valent transition metal-containing molecule being further bound to the titanium oxide complex particles through the linker molecules.

Further, according to the present invention, there is provided a dispersion comprising: the above ultrasonic cancer therapy accelerator; and a solvent in which the ultrasonic cancer therapy accelerator is dispersed.

### BRIEF DESCRIPTION OF DRAWINGS

[FIG. 1] is a diagram showing one embodiment of the ultrasonic cancer therapy accelerator according to the present invention.

[FIG. 2] is a graph showing a fluorescence intensity attributable to the generation of singlet oxygen upon exposure to ultrasonic waves and measured for various particles through a singlet oxygen detecting fluorescence reagent in Example 9.

[FIG. 3] is a graph showing a fluorescence intensity attributable to the generation of hydroxyl radicals upon exposure to ultrasonic waves and measured for titanium oxide complex particles E through a hydroxyl radical detecting fluorescence reagent in Example 11.

[FIG. 4] is a graph showing a fluorescence intensity attributable to the generation of hydroxyl radicals upon exposure to ultrasonic waves and measured for titanium oxide complex particles D and titanium oxide complex particles E through a hydroxyl radical detecting fluorescence reagent in Example 12.

FIG. 5 is a graph showing a fluorescence intensity attributable to the generation of hydroxyl radicals by the addition of hydrogen peroxide and measured for titanium oxide complex particles D and titanium oxide complex particles F through a hydroxyl radical detecting fluorescence reagent in Example 14.

### DESCRIPTION OF EMBODIMENTS

The ultrasonic cancer therapy accelerator according to the present invention includes titanium oxide-metal complex particles comprising titanium oxide particles, a water soluble polymer, linker molecules, and molecules containing a low-valent transition metal. FIG. 1 is a diagram showing one embodiment of an ultrasonic cancer therapy accelerator. As shown in FIG. 1, the ultrasonic cancer therapy accelerator comprises a titanium oxide particle 1 and a water soluble polymer 2 and a low-valent transition metal-containing molecule 4 that are bound to the surface of the titanium oxide particle 1, the low-valent transition metal-containing molecule 4 being bound to the surface of the titanium oxide particle 1 through a linker molecule 3. The binding of the titanium oxide particle 1 to the water soluble polymer 2 and the linker molecule 3 is carried out through at least one functional group selected from the group consisting of carboxyl, amino, diol, salicylate, and phosphate groups.

That is, these functional groups form a strong bond to titanium oxide, and, thus, dispersibility can be maintained despite the high catalytic activity of the titanium oxide particles. Further, the binding of the low-valent transition metal-containing molecule can be maintained through the linker molecule. From the viewpoint of safety in the body, the form of binding in the present invention may be such that the dispersibility is ensured 24 to 72 hours after the administration into the body. A covalent bond is preferred from the viewpoints of stable dispersion under physiological conditions, freedom from the liberation of the water soluble polymer even after ultrasonic irradiation, and little or no damage to normal cells.

Unlike functional groups, such as trifunctional silanol groups, that cause mutual three-dimensional condensation polymerization to entirely cover the surface of the titanium oxide particles by the resultant polymer, it is considered that, the carboxyl, amino, diol, salicylate, and phosphate groups do not cause polymerization between functional groups and, thus, as shown in FIG. 1, exposed parts can be ensured on the surface of the titanium oxide particles. Consequently, the catalytic activity of the titanium oxide particles can be satisfactorily exerted while suppressing the deactivation that may occur due to the covering of the surface of the particles by the polymer.

The water soluble polymer bound to the surface of the titanium oxide particles can allow the antitumor agent according to the present invention to be dispersed even in a near-neutral aqueous solvent, in which the titanium oxide particles cannot be dispersed without difficulties, through the action of charges or hydration. Methods for introducing functional molecules such as antibodies into a water soluble polymer bound to the surface of the titanium oxide particles are known in the art. In order to chemically bind the water soluble polymer to the functional molecules, the water soluble polymer should contain a highly reactive polar group. The polar group contained in the water soluble polymer is lost when the functional molecules are bound. This causes a change in the polarity of the water soluble polymer. That is, it is considered that there is a change in a dispersed balance by charges possessed by the water soluble polymer bound to the surface of the titanium oxide particles or by hydration between before the binding of the functional molecule and after the binding of the functional molecule. The contemplated results can be attained by skillfully regulating the charge or hydration balance involved in the denaturation of the water soluble polymer bound to the surface of the titanium oxide particles. On the other hand, in the present invention, for the low-valent transition metal-containing molecules bound through linker molecules to the surface of the titanium oxide particles, a high level of dispersibility by virtue of the water soluble polymer can be maintained by binding the low-valent transition metal-containing molecules without denaturation of the water soluble polymer. Accordingly, a high degree of freedom is possible in molecule design in binding without considering a change in dispersibility caused by the denauration of the water-soluble polymer.

According to the present invention, an ultrasonic cancer therapy accelerator comprising titanium oxide-metal complex particles, which can maintain a high level of dispersibility without denaturing the water soluble polymer, can be prepared by binding linker molecules to a titanium oxide surface in titanium oxide complex particles dispersed in an aqueous solvent with the use of a water soluble polymer and further binding low-valent transition metal-containing molecules through the linker molecules. The application of ultrasonic waves to the ultrasonic cancer therapy accelerator according to the present invention can provide an antitumor effect attained by the generation of radical species. In general, radical species are highly reactive, but on the other hand, the life time is short and only a small amount of the radicals is dispersed followed by a reaction with adjacent substances. Accordingly, it is considered that, from immediately after the stop of ultrasonic irradiation, the amount of radical species generated until then is significantly reduced. In the ultrasonic cancer therapy accelerator according to the present invention, as described above, the binding of the low-valent transition metal-containing molecule can allow, even after the stop of the ultrasonic irradiation, a Fenton reaction to take place between hydrogen peroxide accumulated in the system by the ultrasonic irradiation and the low-valent transition metal-containing molecules bound to the ultrasonic cancer therapy accelerator and thus can allow radicals to be continuously generated, whereby a persistent antitumor effect can be attained. A high antitumor effect in combination with a persistent effect can be attained by administering the ultrasonic cancer therapy accelerator according to the present invention by intravenous injection into a living body to allow the ultrasonic cancer therapy accelerator to be accumulated at a portion around cancer which is an affected part, and further applying ultrasonic waves. Accordingly, the ultrasonic cancer therapy accelerator according to the present invention can be expected to exert an effect as an agent that accelerates ultrasonic cancer therapy in which, after the administration of an agent, the agent is accumulated at the affected part and the affected part is then irradiated with ultrasonic waves.

Further, how to complex a part of the surface of the titanium oxide particles with an iron-containing crystal or conversely to complex a part of the surface of iron-containing iron oxide particles with a titanium oxide crystal is known in the art. In these conventional methods, disadvantageously, the surface of the titanium oxide particles is covered, or the amount of crystals of titanium oxide is limited. For this reason, it is considered that satisfactorily exerting the catalytic activity of the titanium oxide particles by ultrasonic irradiation is difficult. On the other hand, in the ultrasonic cancer therapy accelerator according to the present invention, strong binding to the surface of the titanium oxide particles can be realized by binding linker molecules containing at least one functional group selected from the group consisting of carboxyl, amino, diol, salicylate, and phosphate groups to the surface of the titanium oxide particles and further binding low-valent transition metal-containing molecules through the linker molecules. Further, as shown in FIG. 1, a number of exposed portions can be ensured on the surface of the titanium oxide particles. As a result, deactivation caused by the covering of the surface of the titanium oxide particles can be suppressed, and the catalytic activity of the titanium oxide particles can be satisfactorily exerted.

According to a preferred embodiment of the present invention, the water soluble polymer used in the present invention is bound to the surface of titanium oxide particles through at least one functional group selected from the group consisting of carboxyl, amino, diol, salicylate, and phosphate groups. This can allow the water soluble polymer to be strongly bound to the surface of the titanium oxide particles. Further, it is considered that, unlike trifunctional silanol or other functional groups that cause mutual three-dimensional condensation polymerization to entirely cover the surface of the titanium oxide particles by the resultant polymer, polymerization between functional groups does not take place and, thus, as shown in FIG. 1, a number of exposed parts can be ensured on the surface of the titanium oxide particles. As a result, the catalytic activity of the titanium oxide particles can be satisfactorily exerted while suppressing the deactivation that may occur due to the covering of the surface of the particles by the polymer.

According to a preferred embodiment of the present invention, the water soluble polymer is not particularly limited as long as the titanium oxide-metal complex particles can be dispersed in an aqueous solvent. Water soluble polymers include anionic or cationic water soluble polymers having charges and nonionic water soluble polymers that do not have charges and impart dispersibility through hydration. At least one of these water soluble polymers is used.

According to a preferred embodiment of the present invention, the water soluble polymer has a weight average molecular weight of 2000 to 100000. The weight average molecular weight of the water soluble polymer is determined by size exclusion chromatography. When the molecular weight falls within the above-defined range, the titanium oxide-metal complex particles can be dispersed by charges possessed by the water soluble polymer or through the action of hydration even in a near-neutral aqueous solvent in which the dispersion of the titanium oxide particles has been regarded as difficult. More preferably, the weight average molecular weight is in the range of 5000 to 100000, more preferably in the range of 5000 to 40000.

In a preferred embodiment of the present invention, any anionic water soluble polymer may be used as long as the ultrasonic cancer therapy accelerator according to the present invention can be dispersed in the aqueous solvent. Anionic water soluble polymers containing a plurality of carboxyl groups include, for example, carboxymethyl starch, carboxymethyl dextran, carboxymethyl cellulose, polycarboxylic acids, and copolymers containing carboxyl group units. Specifically, from the viewpoints of hydrolyzability and solubility of the water soluble polymer, polycarboxylic acids such as polyacrylic acid and polymaleic acid and copolymers of acrylic acid/maleic acid monomers or acrylic acid/sulfonic acid monomers are more preferred, and polyacrylic acid is further preferred.

When polyacrylic acid is used as the anionic water soluble polymer, the weight average molecular weight of polyacrylic acid is 2000 to 100000, more preferably 5000 to 40000, still more preferably 5000 to 20000, from the viewpoint of dispersibility.

In a preferred embodiment of the present invention, any cationic water soluble polymer may be used as long as the ultrasonic cancer therapy accelerator according to the present invention can be dispersed in the aqueous solvent. Cationic water soluble polymers containing a plurality of amino groups include, for example, copolymers comprising polyamino acid, polypeptide, polyamine, and amine units. Specifically, from the viewpoints of hydrolyzability and solubility of the water soluble polymer, polyamines such as polyethylene-imine, polyvinylamine, and polyallylamine are more preferred, and polyethylene-imine is further preferred.

When polyethylene-imine is used as the cationic water soluble polymer, the weight average molecular weight of polyethylene-imine is preferably 2000 to 100000, more preferably 5000 to 40000, still more preferably 5000 to 20000.

In a preferred embodiment of the present invention, any nonionic water soluble polymer may be used as long as the ultrasonic cancer therapy accelerator according to the present invention can be dispersed in the aqueous solvent. Preferred are polymers containing a hydroxyl group and/or a polyoxyalkylene group. Examples of preferred water soluble polymers include polyethylene glycol (PEG), polyvinyl alcohol, polyethylene oxide, dextran, or copolymers containing them. Among them, polyethylene glycol (PEG) and dextran are more preferred, and polyethylene glycol is still more preferred.

When polyethylene glycol is used as the nonionic water soluble polymer, the weight average molecular weight of polyethylene glycol is preferably 2000 to 100000, more preferably 5000 to 40000.

In a preferred embodiment of the present invention, the linker molecules are bound to the surface of the titanium oxide particles. The linker molecules contain at least one functional group selected from the group consisting of carboxyl, amino, diol, salicylate, and phosphate groups.

In a preferred embodiment of the present invention, the linker molecule is a compound comprising a) a saturated or unsaturated chain hydrocarbon group having 6 to 40 carbon atoms, b) a substituted or unsubstituted saturated or unsaturated five- or six-membered heterocyclic group, or c) a substituted or unsubstituted saturated or unsaturated five- or six-membered cyclic hydrocarbon group.

The linker molecules of which the number of carbon atoms is as described above have a smaller molecule size than the water soluble polymer. Further, the linker molecules are bound to the surface of titanium oxide. Accordingly, the titanium oxide-metal complex particles according to the present invention take such a structure that the water soluble polymer is located on an outer shell while the linker molecule is located at an inner position. The outer shell has the largest effect on the dispersibility of the antitumor agent according to the present invention. Specifically, advantageously, the linker molecule located at inner position relative to the water soluble polymer located on the outer shell has a smaller effect on the dispersibility.

The amount of the linker molecule bound to the ultrasonic cancer therapy accelerator according to the present invention is 1.0×10⁻⁶ to 1.0×10⁻³ mol per gram of the titanium oxide particles, more preferably 1.0×10⁻⁶ to 1.0×10⁻⁴ mol per gram of the titanium oxide particles. When the amount of the linker molecule bound to the ultrasonic cancer therapy accelerator is in the above-defined range, advantageously, the ultrasonic cancer therapy accelerator according to the present invention can be dispersed even when a 10% protein solution which is close to an in-vivo environment is used as the solvent. Further, when the amount of the linker molecule bound to the ultrasonic cancer therapy accelerator is in the above-defined range, advantageously, the ultrasonic cancer therapy accelerator according to the present invention, when exposed to ultrasonic waves, exerts catalytic activity and can generate radical species.

Examples of such linker molecules include aromatic compounds and molecules having an alkyl structure. Examples of more specific linker molecules include molecules having a benzene ring, for example, catechols having a catechol structure in molecule thereof, for example, catechol, methyl catechol, tert-butyl catechol dopa, dopamine, dihydroxyphenyl ethanol, dihydroxyphenylpropionic acid, and dihydroxyphenyl acetic acid. Other suitable cyclic molecules include ferrocene, ferrocenecarboxylic acid, ascorbic acid, dihydroxycyclobutenediene, alizarine, and binaphthalenediol. Molecules having an alkyl structure include molecules containing an alkyl group such as a hexyl, octyl, lauryl, palmityl, or stearyl group. Further, molecules containing an alkenyl group such as a hexenyl, octenyl, or oleyl group, or a saturated or unsaturated aliphatic hydrocarbon group such as a cycloalkyl group may also be mentioned.

In a preferred embodiment of the present invention, it is known that, in a molecule containing a low-valent transition metal bound through a linker molecule, the low-valent transition metal decomposes hydrogen peroxide through a Harber-Weiss mechanism to generate hydroxyl radicals (see non-patent document (Kassei Sanso Shu no Kagaku (Chemistry of Active Oxygen Species) [Kikan Kagaku Sosetsu (Quaternaly Chemical Review) No. 7] edited by The Chemical Society of Japan). For example, when divalent iron ions are used as the low-valent transition metal, a well-known Fenton reaction takes place. Various radicals including hydroxyl radicals are cytotoxic. Accordingly, when the low-valent transition metals are bound through the linker molecules, radical can be generated as long as hydrogen peroxide is present, whereby persistent cytotoxic action can be realized. That is, even after the stop of ultrasonic irradiation, a Fenton reaction between hydrogen peroxide accumulated in the system and the low-valent transition metal-containing molecules bound to the antitumor agent according to the present invention can realize persistent generation of more highly oxidative hydroxyl radicals and thus can provide a persistent antitumor effect. It is considered that, when a complex is used as the low-valent transition metal-containing molecule, not only free hydroxyl radicals but also, for example, ferryl complex, which, when an iron complex is used, may be produced, the so-called Crypto-HO. form, participates in the oxidation reaction. Such low-valent transition metals include, in addition to divalent iron, trivalent titanium, divalent chromium, and monovalent copper. Molecules containing such low-valent transition metals include ferrocenecarboxylic acid and a complex of bicinchoninic acid with monovalent copper.

In a preferred embodiment of the present invention, the amount of divalent iron bound through the linker molecules is 1×10⁻⁶ to 1×10⁻³ mol/gram of the titanium oxide particles. The amount of divalent iron bound which is above the upper limit of the above-defined range may reduce the amount of radical species generated and decrease the function as the cancer therapy accelerator. On the other hand, when the amount of iron bound is below the lower limit of the above-defined range, here again, the amount of radical species generated is reduced.

In a preferred embodiment of the present invention, in addition to the low-valent transition metal-containing molecules, other molecules can be contained as the molecules bound through the linker molecules without posing any problem. Such other molecules which may be bound through the linker molecules are not particularly limited. For example, antibody molecules may be bound to actively accumulate the ultrasonic cancer therapy accelerator according to the present invention at a cancer site. An antigen against the antibody is preferably derived from cancer cells or tissues around cancer, such as neovascular vessels. The use of fragments obtained by reducing the molecular weight of the antibody into Fab regions and the like is also possible.

The molecules bound through linker molecules to actively accumulate the antitumor agent according to the present invention at a cancer site are not limited to antibodies and may be, for example, peptides or amino acid sequences that interact with cancer cells or sites derived from tissues around cancer, such as neovascular vessels. More specifically, for example, 5-aminolaevulinic acid, methionine, cysteine, and glycine may be mentioned. The molecules may include sugar chains. Further, the molecules may include binding nucleic acids. The nucleic acid is not particularly limited, and nucleic acid bases such as DNA and RNA, peptide nucleic acids such as PNA, or aptamers in which the nucleic acid base or the peptide nucleic acid forms a higher order structure may also be used.

In a preferred embodiment of the present invention, the linker molecules used in the present invention may be molecules formed by binding, through another linker, a molecule, which imparts the above function, to the functional group bound to the surface of titanium oxide.

In a preferred embodiment of the present invention, possible linkers include, for example, heterobifunctional crosslinkers used in binding biomolecules to each other through different functional groups. Specific examples of linkers include N-hydroxysuccinimide, N-[α-maleimidoacetoxy]succinimide ester, N-[β-maleimidopropyloxy]succinimide ester, N-β-maleimidopropionic acid, N-[β-maleimidopropionic acid]hydrazide•TFA, 1-ethyl-3-[3-dimethylaminopropyl]carbodiimide hydrochloride, N-ε-maleimidocaproic acid, N-[ε-maleimidocaproic acid]hydrazide, N-[ε-maleimidocaproyloxy]succinimide ester, N-[γ-maleimidobutyryloxy]succinimide ester, N-κ-maleimidoundecanoic acid, N-[κ-maleimidoundecanoic acid]hydrazide, succinimidyl-4-[N-maleimidomethyl]-cyclohexane-1-carboxy-[6-amidocaproate], succinimidyl 6-[3-(2-pyridyldithio)-propionamide]hexanoate, m-maleimidobenzoyl-N-hydroxysuccinimide ester, 4-[4-N-maleimidophenyl]butyric acid hydrazide•HCl, 3-[2-pyridyldithio]propionyl hydrazide, N-[p-maleimidophenyl]isocyanate, N-succinimidyl [4-azidophenyl]-1,3′-dithopropionate, N-succinimidyl S-acetylthioacetate, N-succinimidyl S-acetylthiopropionate, succinimidyl 3-[bromoacetamido]propionate, N-succinimidyl iodoacetate, N-succinimidyl [4-iodoacetyl]aminobenzoate, succinimidyl 4-[N-maleimidomethyl]-cyclohexane-1-carboxylate, succinimidyl 4-[p-maleimidophenyl]butyrate, succinimidyl 6-[(β-maleimidopropionamido)hexanoate], 4-succinimidyl oxycarbonyl-methyl-α[2-pyridyldithio]toluene, N-succinimidyl 3-[2-pyridyldithio]propionate, N-[ε-maleimidocaproyloxy]sulfosuccinimide ester, N-[γ-maleimidobutyryloxy]sulfosuccinimide ester, N-[κ-maleimidoundecanoyloxy]-sulfosuccinimide ester, sulfosuccinimidyl 6-[α-methyl-α-(2-pyridyldithio)toluamide]hexanoate, sulfosuccinimidyl 6-[3′-(2-pyridyldithio)-propionamido]hexanoate, m-maleimidobenzoyl-N-hydroxysulfo-succinimide ester, sulfosuccinimidyl [4-iodoacetyl]aminobenzoate, sulfosuccinimidyl 4-[N-maleimidomethyl]-cyclohexane-1-carboxylate, sulfosuccinimidyl 4-[p-maleimidophenyl]butyrate, N-[ε-trifluoroacetylcaproyloxy]succinimide ester, chlorotriazine, dichlorotriazine, and trichlorotriazine. The linker may comprise a plurality of linkers to which other linkers are respectively bound.

In a preferred embodiment of the present invention, the diol group used in binding between the titanium oxide particles and the water soluble polymer and/or the linker molecules is preferably an enediol group, more preferably an α-diol group. When these functional groups are used, highly successful binding to the titanium oxide particles can be realized.

In a preferred embodiment of the present invention, the titanium oxide particles are anatase titanium oxide particles or rutile titanium oxide particles. When catalytic activity exerted upon exposure to ultraviolet light or ultrasonic waves is utilized, anatase titanium oxide is preferred while, when high refractive index or other properties as in cosmetics are utilized, rutile titanium oxide is preferred.

In a preferred embodiment of the present invention, the ultrasonic cancer therapy accelerator has a particle diameter of 20 to 200 nm, more preferably 50 to 200 nm, still more preferably 50 to 150 nm. When the ultrasonic cancer therapy accelerator has the above-defined particle diameter range, the administration of the ultrasonic cancer therapy accelerator into the body of patients to allow the ultrasonic cancer therapy accelerator to reach cancer tumor causes the ultrasonic cancer therapy accelerator to efficiently reach a cancer tissue and to be efficiently accumulated in the cancer tissue by an enhanced permeability and retention effect (EPR effect) as in a drug delivery system. Thereafter, as described above, the application of ultrasonic waves at 400 kHz to 20 MHz causes specific generation of radical species. Accordingly, the cancer tissue can be highly efficiently killed by the ultrasonic irradiation.

In a preferred embodiment of the present invention, when the ultrasonic cancer therapy accelerator has a particle diameter of less than 50 nm (for example, several nanometers), the apparent size can also be increased to attain the EPR effect. That is, a high cancer therapy effect by the EPR effect can be realized by binding semiconductor particles to one another, for example, through a multifunctional linker so that the particles take a form of secondary particles having a particle diameter of 50 to 150 nm.

The particle diameter of the ultrasonic cancer therapy accelerator according to the present invention can be measured by a dynamic light scattering method. Specifically, the particle diameter can be obtained as a value expressed in terms of a Z-average size obtained by a cumulant analysis with a particle size distribution measurement device (Zetasizer Nano, manufactured by Malvern Instruments).

In a preferred embodiment of the present invention, the ultrasonic cancer therapy accelerator according to the present invention is dispersed in a solvent to prepare a dispersion. This allows the ultrasonic cancer therapy accelerator according to the present invention to be used as an ultrasonic cancer therapy accelerator that can be efficiently administered into the body of patients by various methods such as drip infusion, injection, or coating. The liquidity of the dispersion is not limited, and a high level of dispersibility can be realized over a wide pH range of 3 to 10. From the viewpoint of safety in intracorporal injection, preferably, the dispersion has a pH value of 5 to 9, more preferably 5 to 8, and is particularly preferably neutral. In a preferred embodiment of the present invention, the solvent is preferably an aqueous solvent, more preferably a pH buffer solution or physiological saline. The salt concentration of the aqueous solvent is not more than 2 M and is more preferably not more than 200 mM from the viewpoint of safety in intracorporal injection. The content of the ultrasonic cancer therapy accelerator according to the present invention in the dispersion is preferably 0.001 to 1% by mass, more preferably 0.001 to 0.1% by mass. When the ultrasonic cancer therapy accelerator is contained in the above-defined content range, 24 to 72 hr after the administration, the particles can be effectively accumulated at an affected part (tumor). That is, the particles can easily be accumulated at the affected part (tumor), and the dispersibility of the particles in blood can also be ensured. Accordingly, coagulation mass is less likely to be formed, and, thus, there is no possibility that, after the administration, secondary harmful effect such as vessel clogging takes place.

The ultrasonic cancer therapy accelerator according to the present invention can be administered into the body of patients by various methods such as drip infusion, injection, or coating. In particular, the use of the ultrasonic cancer therapy accelerator through an administration route such as intravenous or subcutaneous administration is preferred because burden on patients can be reduced by the so-called DDS-like therapy utilizing EPR effect by the size of particles and the retentivity in blood. The titanium oxide-metal complex particles administered into the body reach the cancer tissue and are accumulated as in the drug delivery system.

Further complexing the ultrasonic cancer therapy accelerator according to the present invention with an antibody or the like followed by use in an administration route through blood vessels, organs or the like located near the affected part is preferred because burden on patients can be reduced by the so-called DDS-like therapy using a high level of dispersbility in in-vivo environments and interaction between an antibody or the like bound to the particles and an antigen derived from the affected part. The titanium oxide-metal complex particles administered into the body reach the cancer tissue and are accumulated as in the drug delivery system.

The ultrasonic cancer therapy accelerator according to the present invention can become cytotoxic upon exposure to ultrasonic waves. The ultrasonic cancer therapy accelerator can kill cells by administrating the ultrasonic cancer therapy accelerator into the body and exposing the ultrasonic cancer therapy accelerator to ultrasonic waves to render the ultrasonic cancer therapy accelerator cytotoxic. The ultrasonic cancer therapy accelerator can kill target cells not only in vivo but also in vitro. In the present invention, the target cells to be killed are not particularly limited but are preferably cancer cells. That is, the ultrasonic cancer therapy accelerator according to the present invention can be used an agent that can be activated upon exposure to ultrasonic waves or ultraviolet light to kill cancer cells.

In a preferred embodiment of the present invention, the cancer tissue in which the ultrasonic cancer therapy accelerator according to the present invention has been accumulated is ultrasonicated. The frequency of ultrasonic waves used is preferably 400 kHz to 20 MHz, more preferably 600 kHz to 10 MHz, still more preferably 1 MHz to 10 MHz. The ultrasonic irradiation time should be properly determined by taking into consideration the position and size of the cancer tissue as a treatment target and is not particularly limited. Thus, the patient's cancer tissue can be killed by ultrasonic waves with high efficiency to realize high cancer therapeutic effect. The ultrasonic waves can reach the deep part in the body from the outside of the body, and the use of the ultrasonic waves in combination with the titanium oxide-metal complex particles according to the present invention can realize the treatment, in a noninvasive state, of an affected part or target site present in the deep part in the body. Further, since the titanium oxide-metal complex particles according to the present invention are accumulated in the affected part or the target site, very weak-intensity ultrasonic waves on such a level that do not adversely affect normal cells around the affected part or target site, can be allowed to act topically only on the place where the titanium oxide-metal complex particles according to the present invention have been accumulated.

The effect of killing cells by activation of the semiconductor particles upon exposure to ultrasonic waves can be realized by generating radical species by ultrasonic irradiation. Specifically, the biological killing effect provided by the semiconductor particles is considered attributable to a qualitative/quantitative increase in radical species. The reason for this is considered as follows. When only ultrasonic irradiation is adopted, hydrogen peroxide and hydroxy radicals are generated in the system. According to the finding by the present inventors, however, the generation of hydrogen peroxide and hydroxy radicals is promoted in the presence of semiconductor particles such as titanium oxide. Further, in the presence of these semiconductor particles, particularly in the presence of titanium oxide, it seems that the generation of superoxide anion and singlet oxygen is promoted. When fine particles on nanometer order are used, the specific generation of these radical species is a phenomenon significantly observed in the ultrasonic irradiation at an ultrasonic irradiation frequency in the range of 400 kHz to 20 MHz, preferably in the range of 600 kHz to 10 MHz, more preferably in the range of 1 MHz to 10 MHz.

### EXAMPLES

The present invention will be further described by the following Examples. In the Examples, “%” is by mass unless otherwise specified.

### Example 1

### Preparation of Polyethylene Glycol-Bound Titanium Oxide Complex Particles

Titanium tetraisopropoxide (3.6 g) and 3.6 g of isopropanol were mixed together, and the mixture was added dropwise to 60 ml of ultrapure water under ice cooling for hydrolysis. After the completion of the dropwise addition, the reaction solution was stirred at room temperature for 30 min. After the stirring, 1 ml of 12 N nitric acid was added dropwise thereto, and the mixture was stirred for peptization at 80° C. for 8 hr. After the completion of the peptization, the reaction solution was filtered through a 0.45 μm filter and was further subjected to solution exchange through a desalination column PD-10 (manufactured by GE Health Care Bioscience) to prepare an acidic titanium oxide sol having a solid content of 1%. The titanium oxide sol was placed in a 100 ml-volume vial bottle, followed by ultrasonication at 200 kHz with an ultrasonic generator MIDSONIC 200 (manufactured by Kaijo Corporation) for 30 min. After the ultrasonication, the average diameter of dispersed particles was measured by dynamic light scattering with Zetasizer Nano ZS (manufactured by Sysmex). Specifically, the ultrasonicated titanium oxide sol was diluted with 12 N nitric acid by a factor of 1000, 0.1 ml of the dispersion was charged into a quartz measurement cell, various parameters of the solvent were set to the same values as water, and the particles diameters of the dispersed particles were measured at 25° C. As a result, it was found that the average diameter of the dispersed particles was 36.4 nm. The titanium oxide sol solution was concentrated at 50° C. using an evaporation dish to finally prepare an acidic titanium oxide sol having a solid content of 20%.

Separately, a mixed solution was prepared from a solution obtained by adding 5 ml of water to 1 g of a copolymer of polyoxyethylene-monoallyl-monomethyl ether with maleic anhydride (average molecular weight: 33659, manufractured by Nippon Oils & Fats Co., Ltd.) and hydrolyzing the mixture and 1-ethyl-3-(3-dimethylaminopropyl)carbodiimide hydrochloride (manufactured by Dojindo) using ultrapure water so that the concentration of the solution and the concentration of 1-ethyl-3-(3-dimethylaminopropyl)carbodiimide hydrochloride were 50 mg/ml and 50 mM, respectively. 4-Aminosalicylic acid (molecular weight Mn=153.14; MP Biomedicals, Inc.) was mixed into the prepared solution to a concentration of 50 mM to prepare a 4-ml solution. A reaction was allowed to proceed at room temperature by shaking/stirring for 72 hr. After the reaction, the resultant solution was transferred to a Spectra/Por CE dialysis tube (fraction molecular weight=3500, Spectrum Laboratories Inc.) and was dialyzed against 4 liters of ultrapure water at room temperature for 24 hr. After the dialysis, the whole solution was transferred to an eggplant flask and was lyophilized overnight. Dimethylformamide (DMF; manufactured by Wako Pure Chemical Industries, Ltd.) (4 ml) was added to and mixed with the resultant powder to prepare a 4-aminosalicylic acid-bound polyethylene glycol solution.

A reaction solution (2.5 ml) was prepared from the 4-aminosalicyclic acid-bound polyethylene glycol solution and the previously obtained anatase-type titanium dioxide sol. In this case, DMF was used to adjust the final concentration of the 4-aminosalicyclic acid-bound polyethylene glycol solution and the final concentration of the anatase-type titanium dioxide sol to 20 (vol/vol) % and 0.25% (on a solid basis), respectively. The reaction solution was transferred to a hydrothermal reaction vessel HU-50 (manufactured by SAN-Al Science Co. Ltd.), and a reaction was allowed to proceed with heating at 80° C. for 6 hr. After the completion of the reaction, the solution was cooled until the temperature of the reaction vessel reached a temperature of 50° C. or below. DMF was removed by an evaporator. Distilled water (1 ml) was added to the residue to prepare a dispersion of polyethylene glycol-bound titanium oxide complex particles. The dispersion was subjected to HPLC [AKTA purifier (manufactured by GE Health Care Bioscience), column:HiPrep 16/60 Sephacryl S-300HR (manufactured by GE Health Care Bioscience), mobile phase: a phosphate buffer solution (pH 7.4), flow rate: 0.3 ml/min]. mobile phase: a phosphate buffer solution) (pH 7.4), flow rate: 0.3 ml/min). As a result, an UV absorption peak was observed in a throughout fraction, and this fraction was collected. The dispersion was diluted with distilled water to prepare an aqueous 0.05 (wt/vol) % solution. The solution was allowed to stand for 72 hr, and the diameter and the zeta potential of dispersed particles were measured by dynamic light scattering with Zetasizer Nano ZS. Specifically, 0.75 ml of the dispersion of the polyethylene glycol-bound titanium oxide complex particles was charged into a zeta potential measurement cell, various parameters of the solvent were set to the same values as water, and the diameters and the zeta potential of the dispersed particles were measured at 25° C. As a result of a cumulant analysis, the diameter of the dispersed particles was found to be 54.2 nm.

### Example 2

### Preparation of Polyacrylic Acid-Bound Titanium Oxide Complex Particles

The procedure of Example 1 was repeated to finally prepare an acidic titanium oxide sol having a solid content of 20%.

The acidic titanium oxide sol (0.6 ml) was diluted with dimethylformamide (DMF) to a volume of 20 ml to disperse the titanium oxide in DMF. A solution (10 ml) of 0.3 g of polyacrylic acid (manufactured by Wako Pure Chemical Industries, Ltd.) having an average molecular weight of 5000 in DMF was added to the dispersion, followed by stirring for mixing. The solution was transferred to a hydrothermal reaction vessel HU-50 (manufactured by SAN-Al Science Co. Ltd.), and a reaction was allowed to proceed with heating at 150° C. for 5 hr. After the completion of the reaction, the solution was cooled until the temperature of the reaction vessel reached a temperature of 50° C. or below. Isopropanol in an amount of two times that of the amount of the reaction solution was added to the reaction solution. The mixture was allowed to stand at room temperature for 30 min and was then centrifuged at 2000 g for 15 min to collect precipitates. The surface of the collected precipitates was washed with ethanol, and 1.5 ml of water was added to obtain a dispersion of polyacrylic acid-bound titanium oxide complex particles. The dispersion was diluted with distilled water by a factor of 100, and the diameter and the zeta potential of dispersed particles were measured by dynamic light scattering with Zetasizer Nano ZS. Specifically, 0.75 ml of the dispersion of the polyacrylic acid-bound titanium oxide complex particles was charged into a zeta potential measurement cell, various parameters of the solvent were set to the same values as water, and the diameters and the zeta potential of the dispersed particles were measured at 25° C. and were found to be 53.6 nm and −45.08 mV, respectively.

### Example 3

### Preparation of Polyethylene Imine-Bound Titanium Oxide Complex Particles

The procedure of Example 1 was repeated to finally prepare an acidic titanium oxide sol having a solid content of 20%.

The titanium oxide sol (3 ml) was dispersed in 20 ml of dimethylformamide (DMF). A solution (10 ml) of 450 mg of polyethylene imine having an average molecular weight of 10000 (manufactured by Wako Pure Chemical Industries, Ltd.) dissolved in DMF was added to the dispersion, followed by stirring for mixing. The solution was transferred to a hydrothermal reaction vessel HU-50 (manufactured by SAN-Al Science Co. Ltd.), and a reaction was allowed to proceed with heating at 150° C. for 5 hr. After the completion of the reaction, the solution was cooled until the temperature of the reaction vessel reached a temperature of 50° C. or below. Acetone in an amount of two times that of the amount of the reaction solution was added to the reaction solution. The mixture was allowed to stand at room temperature for 30 min and was then centrifuged at 2000 g for 15 min to collect precipitates. The surface of the collected precipitates was washed with ethanol, and 1.5 ml of water was added to obtain a dispersion of polyethyleneimine-bound titanium oxide complex particles. The dispersion was diluted with distilled water by a factor of 100, and the diameter and the zeta potential of dispersed particles were measured by dynamic light scattering with Zetasizer Nano ZS. Specifically, 0.75 ml of the dispersion of the polyethyleneimine-bound titanium oxide complex particles was charged into a zeta potential measurement cell, various parameters of the solvent were set to the same values as water, and the diameters and the zeta potential of the dispersed particles were measured at 25° C. and were found to be 57.5 nm and 47.5 mV, respectively.

### Example 4

### Binding of dihydroxyphenylpropionic Acid to Titanium Oxide Complex Particles

The titanium oxide complex particles obtained in Example 1 and dihydroxyphenylpropionic acid were mixed together in ultrapure water according to formulations specified in Table 1, and the total volume of the mixture was brought to 1 ml using the ultrapure water. Titanium oxide complex particles A to C were prepared in the respective compositions.

TABLE 1

| | Material | | | | | |
|---|---|---|---|---|---|---|
| | Titanium oxide | | Titanium oxide | | Titanium oxide | |
| | complex | | complex | | complex | |
| | particles A | | particles B | | particles C | |
| Titanium oxide | 2.5 | wt % | 2.5 | wt % | 0.7 | wt % |
| complex particles | | | | | | |
| Dihydroxyphenyl- | 0.94 | wt % | 0.09 | wt % | 0.01 | wt % |
| propionic acid | | | | | | |
| Ultrapure water | 96.56 | wt % | 97.41 | wt % | 99.29 | wt % |
| Total | 100 | wt % | 100 | wt % | 100 | wt % |

The prepared solutions were allowed to stand at room temperature for 4 hours. For the solutions after the reaction, absorption spectra in a visible light range were confirmed with an ultraviolet-visible spectrophotometer. As a result, an increase in absorbance was observed, suggesting that dihydroxyphenylpropionic acid was bound. Further, the amount of a change in the amount of dihydroxyphenylpropionic acid was determined by confirming a peak at an absorption wavelength of 214 nm by capillary electrophoresis with a photodiode array detector for the solutions before the reaction and the solutions after the reaction under the following conditions.

Apparatus: P/ACE MDQ (manufactured by Beckman Coulter)

Capillary: fused silica capillary 50 μm i.d.×67 cm (effective length 50 cm) (manufactured by Beckman Coulter)

Mobile phase: 50 mM boric acid buffer solution (pH 9.0)

Voltage: 25 kV

Temperature: 20° C.

The amount of dihydroxyphenylpropionic acid bound per mass of the titanium oxide particles was determined based on the amount of the change thus determined. The results are shown in Table 2.

TABLE 2

| | Material | | |
|---|---|---|---|
| | Titanium oxide | Titanium oxide | Titanium oxide |
| | complex | complex | complex |
| | particles A | particles B | particles C |
| Amount of bound | 2.0 × 10⁻⁴ | 5.0 × 10⁻⁵ | 2.0 × 10⁻⁵ |
| dihydroxyphenyl- | | | |
| propionic acid | | | |
| (mol/titanium oxide- | | | |
| gram) | | | |

Further, 1 ml of the solutions were poured into a buffer exchange gravity fall column NAP-10 (GE Health Care Bioscience) and were collected using 1.5 ml of water to remove the unreacted dihydroxyphenylpropionic acid. The removal of dihydroxyphenylpropionic acid, that is, the absence of free dihydroxyphenylpropionic acid, was confirmed by capillary electrophoresis in the same manner as described above. The above experiments revealed that dihydroxyphenylpropionic acid-bound titanium oxide complex particles (titanium oxide complex particles A to C) were successfully prepared.

### Example 5

### Binding of Ferrocenecarboxylic Acid and Dopamine Hydrochloride to Titanium Oxide Complex Particles (Preparation of Titanium Oxide-Metal Complex Particles)

Ferrocenecarboxylic acid (manufactured by Wako Pure Chemical Industries, Ltd.) and dopamine hydrochloride (manufactured by Wako Pure Chemical Industries, Ltd.) were dissolved in dimethylformamide (DMF: manufactured by Wako Pure Chemical Industries, Ltd.) to prepare a 1 mM solution. Likewise, 200 mM benzotriazol-1-yl-oxy-trispyrrolidinophosphonium hexafluorophosphate (PyBop: manufactured by Merck), 200 mM 1-hydroxybenzotriazole (HoBt: manufactured by Dojindo), 20 mM N,N-diisopropylethylamine (DIEA: manufactured by Wako Pure Chemical Industries, Ltd.) were prepared using DMF. Among them, ferrocenecarboxylic acid and dopamine hydrochlorie were mixed followed by dilution with DMF to prepare a 20-ml solution having a concentration that was one-fourth of the original concentration, and the others were mixed followed by dilution with DMF to prepare a 20-ml solution having a concentration that was one-tenth of the original concentration. A reaction was allowed to proceed while gently stirring the mixed solutions at room temperature for 20 hr.

A part of the reaction solutions was diluted with ultrapure water by a factor of 10. The diluted solutions were analyzed by reversed phase chromatography (HPLC system: Prominence (manufactured by Shimadzu Seisakusho Ltd.), column: Chromolith RP-18e 100-3 mm (manufactured by Merck), mobile phase: A methanol (manufactured by Wako Pure Chemical Industries, Ltd.), B 0.1% aqueous trifluoroacetic acid solution (manufactured by Wako Pure Chemical Industries, Ltd.), flow rate: 2 ml/min). In an ultraviolet detector, the wavelength was set to 210 nm, and, after injection (0.02 ml), gradient elution was carried out so that the eluate was 100% ethanol in 1 to 10 min. As a result, a peak considered as a complex of ferrocenecarboxylic acid with dopamine hydrochloride was confirmed. A peak of ferrocenecarboxylic acid alone and a peak of dopamine hydrochloride alone were below the detection limit. These results indicate that a complex of ferrocenecarboxylic acid with dopamine hydrochloride was produced.

The remaining portion of the reaction solution was concentrated by a factor of 10 under the reduced pressure to prepare a concentrated reaction solution. Titanium oxide complex particles obtained in Example 1 were diluted with ultrapure water to a solid content of 1%. The concentrated reaction solution was mixed in an amount of one-tenth of the diluted solution to give a total volume of 1 ml. A reaction was allowed to proceed at room temperature while gently stirring the mixed solution for 1 hr. After the completion of the reaction, the reaction solution was centrifuged (1500 g, 10 min) to separate precipitates, and the supernatant was collected. Further, 1 ml of the solution was poured into a buffer exchange gravity fall column NAP-10 (manufactured by GE Health Care Bioscience) and was collected using 1.5 ml of water to remove the unreacted ferrocenecarboxylic acid-dopamine hydrochloride complex and DMF. For this solution, an absorption spectrum in a visible light range (400 nm) was confirmed with an ultraviolet-visible spectrophotometer (UV1600, manufactured by Shimadzu Seisakusho Ltd.). As a result, an increase in absorbance was observed, suggesting that the ferrocenecarboxylic acid-dopamine hydrochloride complex was bound to the titanium oxide complex particles. These results revealed that titanium oxide-metal complex particles with a ferrocenecarboxylic acid-dopamine hydrochloride complex bound thereto were prepared.

### Example 6

### Binding of Antibody to dihydroxyphenylpropionic Acid-Bound Titanium Oxide-Metal Complex Particles

Dihydroxyphenylpropionic acid-bound titanium oxide-metal complex particles were prepared in quite the same manner as in Example 4, except that the titanium oxide-metal complex particles obtained in Example 5 were used instead of the titanium oxide complex particles obtained in Example 1.

A solution of the titanium oxide-metal complex particles and 1-ethyl-3-(3-dimethylaminopropyl)carbodiimide hydrochloride (manufactured by Dojindo) were mixed using ultrapure water so that the concentration of the titanium oxide-metal complex particles and the 1-ethyl-3-(3-dimethylaminopropyl)carbodiimide hydrochloride in the resultant mixed solution were 20 mg/ml and 80 mM, respectively. A reaction of the mixed solution was allowed to proceed at room temperature for 10 min. The reaction solution was subjected to solution exchange through a desalination column PD-10 (manufactured by GE Health Care Bioscience) with a 20 mM HEPES buffer solution (pH7.4) to prepare a particle solution having a concentration of 20 mg/ml in terms of titanium oxide concentration. An anti-human serum albumin (anti-HSA) monoclonal antibody (mouse IgG:MSU-304, manufactured by Cosmobio) prepared using the same buffer solution as described above was added to the particle solution to a concentration of 3 mg/ml, and the total volume of the solution was brought to 1 ml. A reaction was allowed to proceed at 4° C. for 24 hr, ethanolamine was then added to a final concentration of 0.5 M, and a reaction was allowed to proceed at 4° C. for additional 1 hr. The solution was adjusted to a concentration of 1 mg/ml in terms of titanium oxide concentration, and 1 ml of the adjusted solution was then subjected to HPLC [AKTA purifier (manufactured by GE Health Care Bioscience), column: HiPrep 16/60 Sephacryl S-500HR (manufactured by GE Health Care Bioscience), mobile phase: phosphate buffered physiological saline (pH 7.4), flow rate: 0.3 ml/min]. As a result, an UV absorption peak was observed in a throughout fraction and a fraction in which the anit-HSA monoclonal antibody used in the binding was observed as a simple substance, and these fractions were collected. It was considered from the size of the separated molecules that the throughout fraction was a solution containing antibody molecule-bound titanium oxide-metal complex particles. On the other hand, for the fraction in which the anti-HSA monoclonal antibody was observed as a simple substance, the protein concentration was determined by a Bradford method. As a result, it was found that the concentration of the antibody after the reaction was lower than that of the antibody before the reaction. The above results demonstrate that titanium oxide-metal complex particles with an antibody bound thereto through dihydroxyphenylpropionic acid in dihydroxyphenylpropionic acid-bound titanium oxide-metal complex particles can be prepared.

### Example 7

### Evaluation of Dispersibility of Titanium Oxide Complex Particles

The titanium oxide complex particles (hereinafter referred to as “titanium oxide complex particles D”) obtained in Example 1 and the titanium oxide complex particles A to C obtained in Example 4 were added to a phosphate buffered physiological saline so that the resultant mixture had a solid content of 0.05%. The mixture was allowed to stand at room temperature for 1 hr. Thereafter, the diameters and the zeta potential of dispersed particles were measured by a dynamic light scattering method with Zetasizer Nano ZS in the same manner as in Example 1. The results are shown in Table 3. As a result, it was found that there was no significant change in diameters and zeta potential among the titanium oxide complex particles A to D.

TABLE 3

| | Material | | | |
|---|---|---|---|---|
| | Titanium oxide | Titanium oxide | Titanium oxide | Titanium oxide |
| | complex | complex | complex | complex |
| | particles A | particles B | particles C | particles D |
| Diameter of dispersed | 54.4 | 53.9 | 54.5 | 54.2 |
| particles (nm) | | | | |
| Z potential (mV) | −3.71 | −6.87 | −7.43 | −7.21 |

### Example 8

### Evaluation of Dispersibility of Titanium Oxide-Metal Complex Particles

The titanium oxide-metal complex particles obtained in Example 5 were added to a phosphate buffered physiological saline so that the resultant mixture had a solid content of 0.05%. The mixture was allowed to stand at room temperature for 1 hr. Thereafter, the diameters and the zeta potential of dispersed particles were measured by a dynamic light scattering method with Zetasizer Nano ZS in the same manner as in Example 1. As a result, it was found that the diameter and the zeta potential of the dispersed particles were 52.5 nm and −4.48 mV, respectively, that is, were not significantly different from the measurement results of the diameter and zeta potential in Example 7.

### Example 9

### Evaluation of Capability of Titanium Oxide Complex Particles to Generate Singlet Oxygen Upon Exposure to Ultrasonic Waves

The titanium oxide complex particles (hereinafter referred to as “titanium oxide complex particles D”) obtained in Example 1 and the titanium oxide complex particles A to C obtained in Example 4 were added to a phosphate buffered physiological saline so that the resultant mixture had a solid content of 0.05%. Separately, a solution consisting of a phosphate buffered physiological saline alone was provided as a control. Singlet Oxygen Sensor Green reagent (manufactured by Molecular Probes) which is a reagent for determining the generation of singlet oxygen was mixed into 3 ml of each of the solutions according to a manufacturer's instruction manual to prepare testing solutions. The testing solutions were irradiated with ultrasonic waves by an ultrasonic irradiator (ULTRASONIC APPARATUS ES-2:1MHz manufactured by OG GIKEN CO., LTD.) for 3 min under conditions of 0.4 W/cm² and 50% duty cycle operation. Each solution (400 μl) was extracted as a measurement sample before and after the irradiation. For each measurement sample, the fluorescence intensity at Ex=488 nm and Em=525 nm attributable to singlet oxygen generation was measured with a fluorescence spectrophotometer (RF-5300PC, manufactured by Shimadzu Seisakusho Ltd.). The results were as shown in FIG. 2. As shown in FIG. 2, it was found that, upon exposure to the ultrasonic waves, the titanium oxide complex particles A to D could generate singlet oxygen more efficiently than the control. Further, it was considered that the generation of the singlet oxygen decreased with an increase in the amount of the linker bound per mass of the titanium oxide particles.

### Example 10

### Binding of dihydroxyphenylpropionic Acid to Titanium Oxide Complex Particles (2)

The titanium oxide complex particles and the dihydroxyphenylpropionic acid obtained in Example 1 were mixed together in 1) a 20 mmol/liter acetic acid-sodium acetate buffer solution (pH=3.6), 2) a 20 mmol/liter MES buffer solution (manufactured by Dojindo; pH=6.0), and 3) a 20 mmol/liter HEPES buffer solution (manufactured by Dojindo; pH=8.1) so that the final concentration of the titanium oxide complex particles and the final concentration of the dihydroxyphenylpropionic acid in the resultant mixed solutions were 2% and 50 mmol/liter, respectively. The total volume of each of the mixed solution was adjusted to 0.8 ml.

The adjusted solutions were stirred at 40° C. for 25 hr. For each solution, an absorption spectrum was measured in an ultraviolet-visible range (200 to 600 nm) with an ultraviolet-visible spectrometer. For the solution into which only dihydroxyphenylpropionic acid was mixed, 1) in the 20 mmol/liter acetic acid-sodium acetate buffer solution (pH=3.6), little or no change was observed in the absorption spectrum as compared with the absorption spectrum measured zero hr after the preparation, whereas, 2) in the 20 mmol/liter MES buffer solution (pH=6.0) and in 3) 20 mmol/liter HEPES buffer solution (pH=8.1), a change in an absorption spectrum was observed as compared with the absorption spectrum measured zero hr after the preparation and a change in color to a light red color was also visually confirmed. It was considered from these results that dihydroxyphenylpropionic acid caused a change and was unstable at pH 6.0 or higher. For the solution into which the titanium oxide complex particles and dihydroxyphenylpropionic acid were mixed, 1) in the 20 mmol/liter acetic acid-sodium acetate buffer solution (pH=3.6), a change in an absorption spectrum was confirmed as compared with the absorption spectrum measured zero hr after the preparation and a change in color to a deep brown color was also visually confirmed. Since no significant change was observed when only dihydroxyphenylpropionic acid was mixed, this change was considered to be attributable to charge migration as a result of binding of dihydroxyphenylpropionic acid to the titanium oxide complex particles.

Next, 1) in the 20 mmol/liter acetic acid-sodium acetate buffer solution (pH=3.6), for the solutions zero hr and 25 hr after the preparation, the amount of a change in dihydroxyphenylpropionic acid was determined by confirming a peak at an absorption wavelength of 214 nm with a photodiode array detector under the following conditions by capillary electrophoresis.

Apparatus: P/ACE MDQ (manufactured by Beckman Coulter)

Capillary: fused silica capillary 50 μm i.d.×67 cm (effective length 50 cm) (manufactured by Beckman Coulter)

Mobile phase: 50 mM boric acid buffer solution (pH 9.0)

Voltage: 25 kV

Temperature: 20° C.

The amount of dihydroxyphenylpropionic acid bound per mass of the titanium oxide particles in 1) 20 mmol/liter acetic acid-sodium acetate buffer solution (pH=3.6) was determined based on the amount of the change thus determined and was found to be 7.7×10⁻⁴ mol of dihydroxyphenylpropionic acid/g of titanium oxide particles.

### Example 11

### Evaluation of Capability of Titanium Oxide-Metal Complex Particles to Generate Hydroxyl Radials Upon Exposure to Ultrasonic Waves

The titanium oxide-metal complex particles with a ferrocenecarboxylic acid-dopamine hydrochloride complex bound thereto obtained in Example 5 (hereinafter referred to as “titanium oxide complex particles E”) were added to phosphate buffered physiological saline (pH 7.4) to prepare a solution having a solid content of 0.05%. Separately, a solution consisting of a phosphate buffered physiological saline (pH 7.4) alone was provided as a control. Each of the solution (3 ml) was provided as a testing solution. The solutions were irradiated with ultrasonic waves with an ultrasonic irradiator (ULTRASONIC APPARATUS ES-2: 1 MHz, manufactured by OG GIKEN CO., LTD.) for 3 min under conditions of 0.4 W/cm² and 50% pulse. After the irradiation, Hydroxyphenyl Fluorescein (HPF, manufactured by DAI ICHI PURE CHEM CO. LTD.) which is a reagent for determining the generation of hydroxyl radicals was mixed into each irradiated solution according to the manufacturer's instruction, and the mixtures were allowed to stand at room temperature for 15 min and 30 min. For each standing time, 400 μl of each solution before and after the irradiation was extracted as a measurement sample. For each measurement sample, the fluorescence intensity at Ex=490 nm and Em=515 nm attributable to the generation of hydroxyl radicals was measured with a fluorescence spectrophotometer (RF-5300PC, manufactured by Shimadzu Seisakusho Ltd.). The results were as shown in FIG. 3. As shown in FIG. 3, it was found that, upon exposure to the ultrasonic waves, the titanium oxide complex particles E could generate hydroxyl radicals more efficiently than the control. Further, for the titanium oxide complex particles E, the fluorescence value increased with an increase in standing time. Accordingly, it was considered that hydroxyl radicals were continuously generated.

### Example 12

### Evaluation of Capability of Titanium Oxide Complex Particles and Titanium Oxide-Metal Complex Particles to Generate Hydroxyl Radials Upon Exposure to Ultrasonic Waves

The titanium oxide complex particles (designated as “titanium oxide complex particles D”) obtained in Example 1 and the titanium oxide-metal complex particles with the ferrocenecarboxylic acid-dopamine hydrochloride complex bound thereto (designated as “titanium oxide complex particles E” obtained in Example 5 were added to a phosphate buffered physiological saline (pH 7.4) to prepare solutions having a solid content of 0.05%. Separately, a solution consisting of a phosphate buffered physiological saline (pH 7.4) alone was provided as a control. Each of the solution (3 ml) was provided as a testing solution. The solutions were irradiated with ultrasonic waves with an ultrasonic irradiator (ULTRASONIC APPARATUS ES-2: 1 MHz, manufactured by OG GIKEN CO., LTD.) for 3 min under conditions of 0.4 W/cm² and 50% pulse. After the irradiation, Hydroxyphenyl Fluorescein (HPF, manufactured by DAI ICHI PURE CHEM CO. LTD.) which is a reagent for determining the generation of hydroxyl radicals was mixed into each irradiated solution according to the manufacturer's instruction, and the mixtures were allowed to stand at room temperature for 30 min. For the standing time, 400 μl of each solution before and after the irradiation was extracted as a measurement sample. For each measurement sample, the fluorescence intensity at Ex=490 nm and Em=515 nm attributable to the generation of hydroxyl radicals was measured with a fluorescence spectrophotometer (RF-5300PC, manufactured by Shimadzu Seisakusho Ltd.). The results were as shown in FIG. 4. As shown in FIG. 4, it was found that, upon exposure to the ultrasonic waves, the titanium oxide complex particles D and the titanium oxide complex particles E could generate hydroxyl radicals more efficiently than the control. Further, the amount of hydroxyl radicals generated by the titanium oxide complex particles E was larger than the amount of hydroxyl radicals generated by the titanium oxide complex particles D. These results revealed that the titanium oxide-metal complex particles could increase the amount of hydroxyl radicals generated upon exposure to ultrasonic waves.

### Example 13

### Binding of ferrocenecarboxylic Acid and Dopamine Hydrochloride to Titanium Oxide Complex Particles (Preparation of Titanium Oxide-Metal Complex Particles)

Ferrocenecarboxylic acid (manufactured by Wako Pure Chemical Industries, Ltd.) and dopamine hydrochloride (manufactured by Wako Pure Chemical Industries, Ltd.) were dissolved in dimethylformamide (DMF: manufactured by Wako Pure Chemical Industries, Ltd.) to prepare a 5 mM solution. Likewise, 200 mM benzotriazol-1-yl-oxy-trispyrrolidinophosphonium hexafluorophosphate (PyBop: manufactured by Merck), 200 mM 1-hydroxybenzotriazole (HoBt: manufactured by Dojindo), 40 mM N,N-diisopropylethylamine (DIEA: manufactured by Wako Pure Chemical Industries, Ltd.) were prepared using DMF. Among them, ferrocenecarboxylic acid and dopamine hydrochlorie were mixed followed by dilution with DMF to prepare an 8-ml solution having a concentration that was one-fourth of the original concentration, and the others were mixed followed by dilution with DMF to prepare an 8-ml solution having a concentration that was one-eighth of the original concentration. A reaction was allowed to proceed while gently stirring the mixed solutions at room temperature for 20 hr.

A part of the reaction solutions was diluted with ultrapure water by a factor of 10. The diluted solutions were analyzed by reversed phase chromatography (HPLC system: Prominence (manufactured by Shimadzu Seisakusho Ltd.), column: Chromolith RP-18e 100-3 mm (manufactured by Merck), mobile phase: A methanol (manufactured by Wako Pure Chemical Industries, Ltd.), B 0.1% aqueous trifluoroacetic acid solution (manufactured by Wako Pure Chemical Industries, Ltd.), flow rate: 2 ml/min). In an ultraviolet detector, the wavelength was set to 210 nm, and, after injection (0.02 ml), gradient elution was carried out so that the eluate was 100% ethanol in 1 to 10 min. As a result, a peak considered as a complex of ferrocenecarboxylic acid with dopamine hydrochloride was confirmed. A peak of ferrocenecarboxylic acid alone and a peak of dopamine hydrochloride alone were below the detection limit. These results indicate that a ferrocenecarboxylic acid-dopamine hydrochloride complex was produced.

The remaining portion of the reaction solution was concentrated under the reduced pressure, and the concentrate was diluted with DMF to prepare a 1-ml concentrated reaction solution. Titanium oxide complex particles obtained in Example 1 was diluted with DMF to a solid content of 0.625%. The concentrated reaction solution was mixed in amounts of one-tenth, one-thirtieth, and one-ninetieth and the mixtures were diluted with DMF to give a total volume of 3 ml. A reaction was allowed to proceed at room temperature while gently stirring the mixed solutions for 5 hr. After the completion of the reaction, the reaction solutions were dried under the reduced pressure, and about 1 ml of ultrapure water was added to the residue. The resultant precipitates were centrifuged (1500 g, 10 min), and the supernatants were collected. Further, 1 ml of each of the supernatants was transferred to a centrifugal membrane separation apparatus Amicon Ultra-15 (MWCO=100000, manufactured by Millipore Corporation). Ultrapure water (14 ml) was added, and the mixture was centrifuged (1500 g, 15 min) to remove the filtrate. The centrifugal filtration was repeated fix times to remove the unreacted ferrocenecarboxylic acid-dopamine hydrochloride complex and DMF. These solutions were diluted with ultrapure water to a solid content of 0.5%, and, for the diluted solutions, an absorption spectrum in a visible light range (400 nm) was confirmed with an ultraviolet-visible spectrophotometer (UV1600, manufactured by Shimadzu Seisakusho Ltd.). As a result, it was found that the absorbance increased depending upon the amount of the mixed ferrocenecarboxylic acid-dopamine hydrochloride complex, suggesting that the ferrocenecarboxylic acid-dopamine hydrochloride complex was bound to the titanium oxide complex particles. These results revealed that titanium oxide-metal complex particles with a ferrocenecarboxylic acid-dopamine hydrochloride complex bound thereto were prepared.

### Example 14

### Evaluation of Capability of Titanium Oxide-Metal Complex Particles to Generate Hydroxyl Radials by Addition of Hydrogen Peroxide

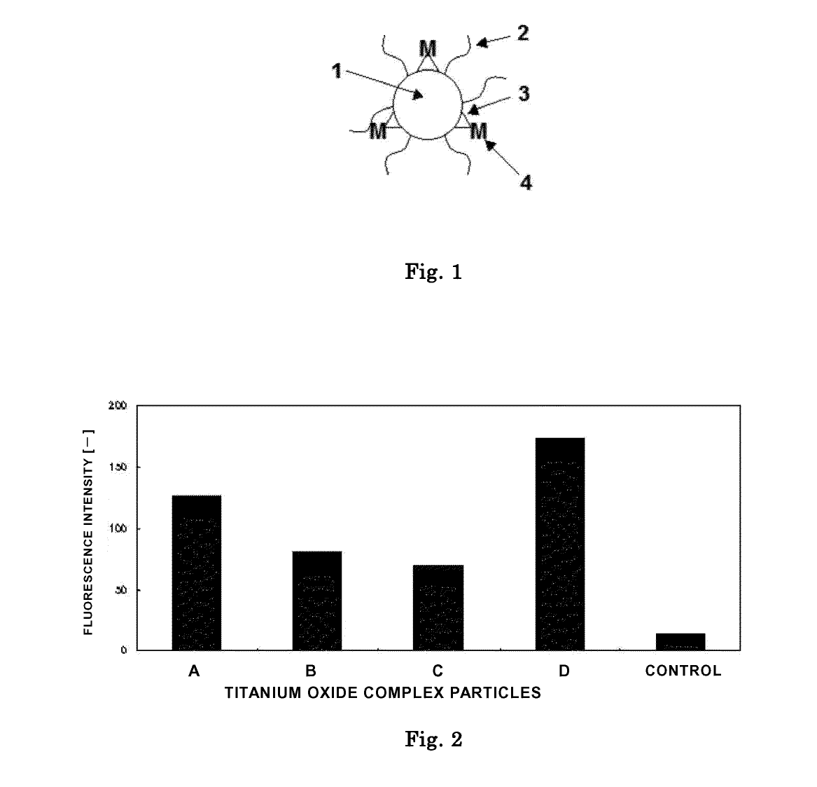

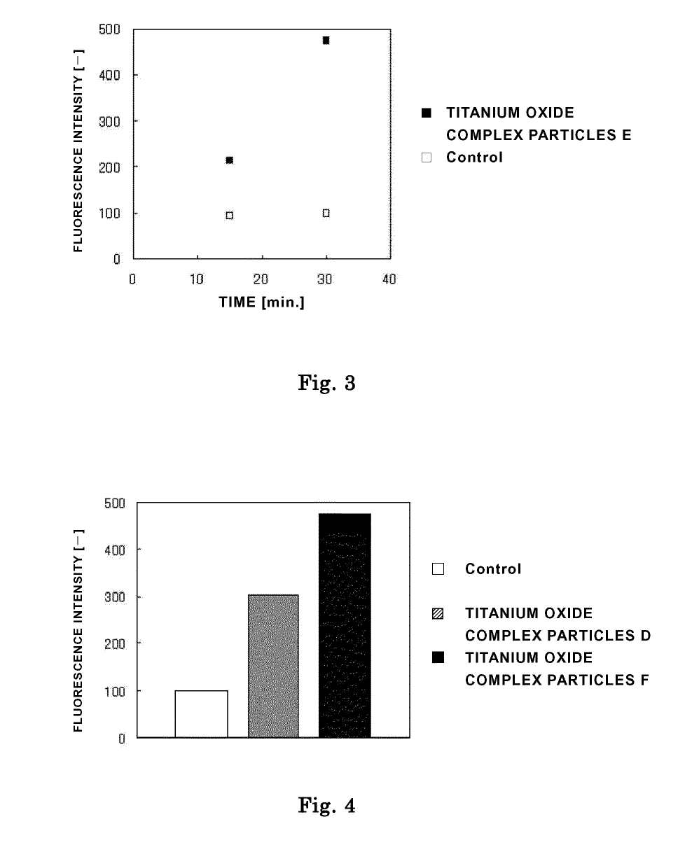

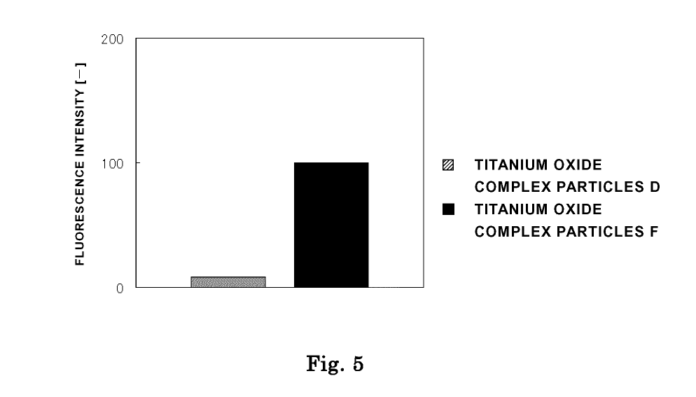

The titanium oxide complex particles (designated as “titanium oxide complex particles D”) obtained in Example 1 and the titanium oxide-metal complex particles obtained by mixing the concentrated reaction solution in an amount of one-ninetieth in Example 13 (designated by “titanium oxide complex particles F”) were mixed with ultrapure water so that the resultant solutions had a solid content of 1.0%. Thereafter, 0.05 ml of a ten-fold concentration solution of phosphate buffered physiological saline (pH7.4), 0.15 ml of ultrapure water, and 0.1 ml of 10 mM hydrogen peroxide (manufactured by Wako Pure Chemical Industries, Ltd.) were mixed into 0.2 ml of each of the solution of the titanium oxide complex particles D and the solution of the titanium oxide complex particles F. Immediately after the mixing, Hydroxyphenyl Fluorescein (HPF, manufactured by DAI ICHI PURE CHEM CO. LTD. which is a reagent for determining the generation of hydroxyl radicals was mixed into the mixtures according to the manufacturer's instruction to prepare measurement samples. For each measurement sample, the fluorescence intensity at Ex=490 nm and Em=515 nm attributable to the generation of hydroxyl radicals was measured with a fluorescence spectrophotometer (RF-5300PC, manufactured by Shimadzu Seisakusho Ltd.). The measurement was carried out immediately after the mixing and 40 min after the mixing. The results were as shown in FIG. 5. It was confirmed that the titanium oxide complex particles F, when mixed with hydrogen peroxide, can generate hydroxyl radicals more efficiently than the titanium oxide complex particles D. These results revealed that the titanium oxide-metal complex particles could increase the amount of hydroxyl radicals generated in the presence of hydrogen peroxide.

### Example 15

### Evaluation of Dispersion Stability of Titanium Oxide-Metal Complex Particles in Protein Solution

The titanium oxide-metal complex particles-containing dispersion obtained in Example 13 by mixing the concentrated reaction solution in an amount of one-tenth was added to an F12 medium (manufactured by GIBCO) containing 10 (vol/vol) % of fetal calf serum (manufactured by Japan Bio Serum) so that the final concentration was 0.05 (wt/vol) %. The mixture was allowed to stand at room temperature for 1 hr and 18 hr. For each standing time, the diameter of the dispersed particles was measured with Zetasizer Nano ZS (manufactured by Sysmex) in the same manner as in Example 1. As a result, it was found that the diameter of dispersed particles 1 hr after the start of the standing and the diameter of dispersed particles 18 hr after the start of the standing were 52.9 nm and 54.0 nm, respectively. Thus, the diameter of titanium oxide-metal complex particles dispersed in the protein solution remained substantially unchanged, indicating stable dispersibility of the titanium oxide-metal complex particles.

1. Titanium oxide-metal complex particles that have catalytic activity upon exposure to ultrasonic waves, comprising:
titanium oxide complex particles comprising titanium oxide particles and a water soluble polymer bound to the surface of the titanium oxide particles through at least one functional group selected from the group consisting of carboxyl, amino, diol, salicylate and phosphate groups; and
linker molecules further bound to the surface of the titanium oxide complex particles, the linker molecules being a compound
(1) having at least one functional group selected from the group consisting of carboxyl, amino, diol, salicylate and phosphate groups and
(2) comprising a) a saturated or unsaturated chain hydrocarbon group having 6 to 40 carbon atoms, b) a substituted or unsubstituted saturated or unsaturated five- or six-membered heterocyclic group, or c) a substituted or unsubstituted saturated or unsaturated five- or six-membered cyclohydrocarbon group, the functional groups in the compound not being mutually polymerized, the compound being bound to the titanium oxide through the functional group,
a low-valent transition metal-containing molecule being further bound to the titanium oxide complex particles through the linker molecules.

2. The titanium oxide-metal complex particles according to claim 1, wherein the amount of the linker molecules bound per mass of the titanium oxide particle is 1×10⁻⁶ to 1×10⁻³ mol/titanium oxide particle-gram.

3. The titanium oxide-metal complex particles according to claim 1, wherein the linker molecule is a catechol compound.

4. The titanium oxide-metal complex particles according to claim 3, wherein the linker molecule is at least one compound selected from the group consisting of dopamine and dihydroxyphenylpropionic acid.

5. The titanium oxide-metal complex particles according to claim 1, wherein the molecule containing the low-valent transition metal contains divalent iron.

6. The titanium oxide-metal complex particles according to claim 5, wherein the amount of the divalent iron bound per mass of the titanium oxide particle is 1×10⁻⁶ to 1×10⁻³ mol/titanium oxide particle-gram.

7. The titanium oxide-metal complex particles according to claim 5, wherein the molecule containing the low-valent transition metal is ferrocenecarboxylic acid.

8. The titanium oxide-metal complex particles according to claim 1, wherein the water soluble polymer has a weight average molecular weight of 5000 to 40000.

9. The titanium oxide-metal complex particles according to claim 1, wherein the water soluble polymer comprises at least one polymer selected from the group consisting of polyethylene glycol, polyacrylic acid, and polyethylene-imine.

10. The titanium oxide-metal complex particles according to claim 1, which has a particle diameter of 20 to 200 nm.

11. A dispersion comprising: the titanium oxide-metal complex particles according to claim 1; and a solvent in which the titanium oxide-metal complex particles are dispersed.

12. The dispersion according to claim 11, wherein the solvent is an aqueous solvent.

13. The dispersion according to claim 11, wherein the solvent has pH 5 to 8.

14. The dispersion according to claim 11, wherein the solvent is physiological saline.

15. The dispersion according to claim 11, wherein the ultrasonic cancer therapy accelerator is contained in an amount of 0.001 to 1% by mass.

16. A method for killing a cell comprising the steps of contacting the titanium oxide-metal complex particles according to claim 1 with a target cell and irradiating ultrasonic waves to the titanium oxide-metal complex particles with the target cell.

17. The method according to claim 16, which is carried out in vivo or in vitro.

18. The method according to claim 16, wherein the target cell is a cancer cell.

19. A method for treating cancer comprising the steps of administrating the titanium oxide-metal complex particles according to claim 1 to a mammal having cancer tissue and irradiating ultrasonic wave to the cancer tissue of the mammal.
